(19) United States
(12) Patent Application Publication
(10) Pub. No.: US 2025/0049746 A1
(43) Pub. Date: Feb. 13, 2025

(54) Medicinal Oil for Reducing Gastrointestinal Reaction Caused by Sulforaphane (SFA) and Sulforaphene

(71) Applicant: Beijing University of Chemical Technology, Beijing (CN)

(72) Inventors: Qipeng YUAN, Beijing (CN); Pengtao LIU, Beijing (CN); Li CHENG, Beijing (CN)

(21) Appl. No.: 18/288,798

(22) PCT Filed: Sep. 8, 2022
(86) PCT No.: PCT/CN2022/117835

§ 371 Date: Oct. 27, 2023

(30) Foreign Application Priority Data
Sep. 8, 2021 (CN) 202111047989.8

(51) Int. Cl. A61K 31/26; A61K 9/48; A61K 36/31

(52) U.S. Cl. CPC A61K 31/26; A61K 9/4825; A61K 9/4875; A61K 36/31

(57) ABSTRACT

The present disclosure provides a preparation method of a medicinal oil for reducing a gastrointestinal reaction caused by sulforaphane (SFA) and sulforaphene. In the present disclosure, the medicinal oil includes the SFA and the sulforaphene that serve as active ingredients, and the SFA and the sulforaphene each have a weight percentage content of 0.1% to 50%. An auxiliary material vegetable oil for preparing the medicinal oil is one or a mixture of two or more selected from the group consisting of soybean oil, corn oil, peanut oil, sesame oil, cottonseed oil, linseed oil, castor oil, rapeseed oil, sunflower seed oil, camellia seed oil, and olive oil. The SFA, the sulforaphene, and the auxiliary material are mixed to prepare a capsule. The medicinal oil can be used to reduce the gastrointestinal reaction caused by the SFA and the sulforaphene.

### TECHNICAL FIELD

The present disclosure relates to a preparation method of a medicinal oil for reducing a gastrointestinal reaction caused by sulforaphane (SFA) and sulforaphene. The medicinal oil includes the SFA and the sulforaphene that serve as active ingredients, uses a vegetable oil as a main auxiliary material, and adopts a capsule as a dosage form.

### BACKGROUND

Cancer has seriously threatened human life and health due to a high mortality rate. An epidemiological study in the Mediterranean region has showed that some residents who regularly consume cruciferous vegetables have a lower risk of developing cancer, indicating that there are one or more active ingredients in cruciferous plants that can inhibit the growth of tumor cells. Further researches have confirmed that these active ingredients belong to isothiocyanate compounds. The most studied and representative isothiocyanate compound is sulforaphane (SFA), which is isolated from cauliflower. There have been dozens of clinical trials on SFA around the world, but no drug based on the SFA has been marketed due to its severe gastrointestinal reactions. Like the SFA, sulforaphene belongs to an isothiocyanate compound, shows a similar anti-cancer activity, and can cause similar severe gastrointestinal reactions.

### SUMMARY

It can be seen from the background that SFA has desirable druggability, but causes severe gastrointestinal reactions to prevent patients from taking larger doses of the SFA. This has resulted in an inability of the SFA to reach an effective dose in multiple phase-II clinical trials without reaching its toxic dose. Therefore, reducing the gastrointestinal reactions of SFA and increasing the dosage of patients can further improve an efficacy of the SFA. Moreover, a similar situation is also shown for sulforaphene in the preliminary trials: some patients stop taking the drug in advance due to severe gastrointestinal reactions, such that the sulforaphene fails to achieve satisfactory clinical results in the end.

In order to solve the gastrointestinal reactions caused by SFA and sulforaphene, the inventors have tried various methods. It has been found through sustained-release and other methods that as long as the SFA and sulforaphene are not absorbed in the stomach, the gastrointestinal reactions caused by same are greatly reduced. As a result, a vegetable oil is eventually selected to encapsulate the SFA and sulforaphene to prepare a medicinal oil, and then the medicinal oil is made into a capsule, such that the two drugs can be disintegrated in the intestinal tract and then absorbed in the small intestine. This process not only maintains the desired concentration of the two drugs, but also greatly reduces the gastrointestinal reactions.

The present disclosure mainly relates to the following aspects:
1. The present disclosure provides a medicinal oil for reducing a gastrointestinal reaction caused by SFA and sulforaphene, further including at least a vegetable oil, where the SFA or/and the sulforaphene serve as active ingredients of the medicinal oil, and the SFA and the sulforaphene each have a weight percentage content of 0.1% to 50%.
2. For the medicinal oil, the SFA and the sulforaphene each are selected from the group consisting of a natural product extract, a biological synthesis product, and a chemical synthesis product.
3. For the medicinal oil, the natural product extract is extracted from the group consisting of Brassica oleracea var. gemmifera Zenker, Brassica oleracea, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton.
4. For the medicinal oil, the vegetable oil is one or a mixture of two or more selected from the group consisting of soybean oil, corn oil, peanut oil, sesame oil, cottonseed oil, linseed oil, castor oil, rapeseed oil, sunflower seed oil, camellia seed oil, and olive oil.
5. For the medicinal oil, the medicinal oil is a capsule, and the capsule is selected from the group consisting of a hard capsule or a soft capsule.
6. For the medicinal oil, the capsule is selected from the group consisting of a sustained-release capsule, a controlled-release capsule, and an enteric-coated capsule that are loaded with a sustained-release material and a normal capsule that is not loaded with the sustained-release material.
7. For the medicinal oil, the capsule is orally administered.
8. For the medicinal oil, the medicinal oil further includes one or more selected from the group consisting of a lubricant, a wetting agent, a sweetener, a flavoring agent, an emulsifier, a suspending agent, and a preservative.
9. For the medicinal oil, the medicinal oil can be used to reduce the gastrointestinal reaction SFA and sulforaphene.

The present disclosure relates to a preparation method of a medicinal oil for reducing a gastrointestinal reaction caused by SFA and sulforaphene. In the present disclosure, the medicinal oil includes the SFA and sulforaphene that serve as active ingredients, an auxiliary material used to prepare the medicinal oil is a vegetable oil, and the SFA, the sulforaphene, and the auxiliary material are mixed to prepare a capsule. The medicinal oil shows an extremely significant effect in reducing the gastrointestinal reactions caused by SFA and sulforaphene.

(1) After oral administration of SFA and sulforaphene tablets at a dose of 30 mg/person/time, more than half of the patients have experienced abdominal pain and flatulence to varying degrees within 10 min, and a small number of subjects have experienced heartburn: these symptoms have gradually alleviated 1 h after taking the drug. and are basically no longer felt after 2 h.

(2) After the subjects have took SFA and sulforaphene capsules orally at a dose of 30 mg/person/time, less than one-tenth of the patients feel flatulent within 10 min; no subject reports heartburn, and the flatulence disappears within 30 min.

In summary, the medicinal oil of the present disclosure is applicable to a wide range of people and is effective on most subjects, and can significantly increase the dosage of SFA and sulforaphene. thereby further increasing the activities of SFA and sulforaphene.

### BRIEF DESCRIPTION OF THE DRAWINGS

FIG. 1 shows an electron microscope image of the sulforaphene in a vegetable oil: and

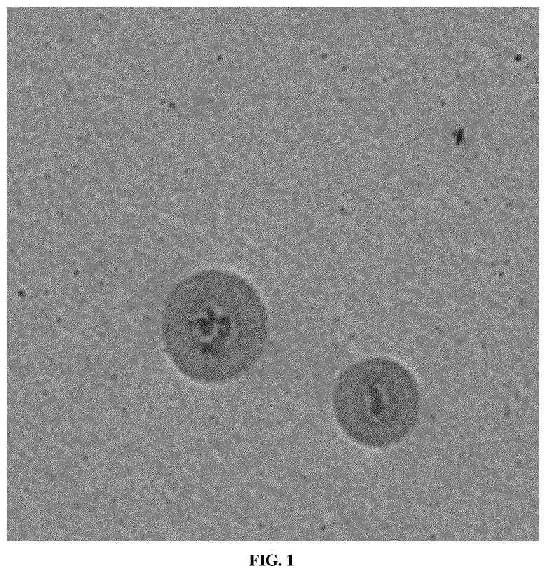

FIG. 2 shows an electron microscope image of the SFA in the vegetable oil.

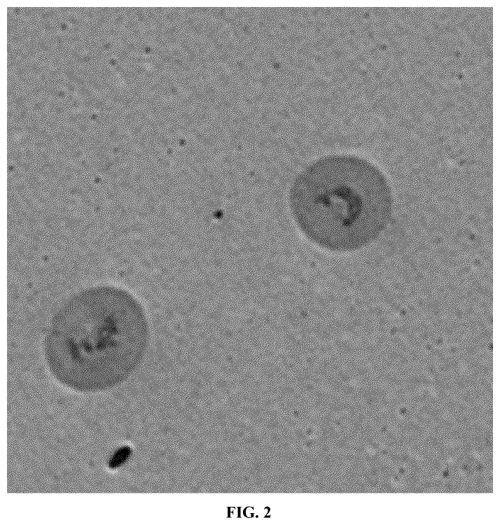

### DETAILED DESCRIPTION OF THE EMBODIMENTS

The present disclosure relates to a preparation method of a medicinal oil for reducing a gastrointestinal reaction caused by SFA and sulforaphene. In the present disclosure, the medicinal oil includes the SFA and sulforaphene that serve as active ingredients. an auxiliary material used to prepare the medicinal oil is a vegetable oil, and the SFA, the sulforaphene, and the auxiliary material are mixed to obtain a capsule. The medicinal oil shows an extremely significant effect in reducing the gastrointestinal reactions caused by SFA and sulforaphene.

In the present disclosure, the SFA and the sulforaphene can be prepared by any method known in the art, such as natural product extraction, biological synthesis, or chemical synthesis, as long as the SFA and the sulforaphene extracts have the effects of preventing. improving. and treating leukotrichia and alopecia. In the present disclosure, the SFA and the sulforaphene are preferably extracted from a natural product extract, and the natural product extract is derived from the group consisting of Brassica oleracea var. gemmifera Zenker, Brassica oleracea, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton.

In a specific example of the present disclosure, a preparation method of the SFA and the sulforaphene includes the following steps:
(1) crushing or homogenizing radish seeds, radish seed sprouts or broccoli seeds, adding 5 to 20 times a volume of tap water, deionized water, or a buffer solution with a pH value of 5.0 to 8.0, stirring at 5° C. to 50° C. to allow hydrolysis 10 min to 300 min, adding hydrochloric acid to adjust a pH value of a resulting hydrolyzate to 1.0 to 3.0, allowing to stand overnight, and conducting vacuum filtration or centrifugation to obtain SFA and sulforaphene hydrolyzates;
(2) extracting the SFA and sulforaphene hydrolyzates obtained in step (1) 2 to 5 times with an organic solvent that is added in an amount 0.5 to 5 times a volume of the SFA and sulforaphene hydrolyzates, recovering the SFA and sulforaphene in the hydrolyzate, collecting an extraction layer in the organic solvent, distilling the extraction layer under reduced pressure to obtain crude extracts of the SFA and sulforaphene, while recovering the organic solvent; where the organic solvent is an organic solvent commonly used in this field, such as n-hexane, cyclohexane, diethyl ether, n-butanol, chloroform, dichloromethane, or ethyl acetate;
(3) adding the crude extracts of the SFA and sulforaphene obtained in step (2) to a molecular distillation device to allow first-stage molecular distillation to remove residual organic solvents, moisture, and low-boiling point impurities in the crude extracts of the SFA and sulforaphene, collecting a heavy component flowing out from a distillation wall of a first-stage molecular distillation device to obtain a first-stage molecular distillation heavy component; where the conditions for the first-stage molecular distillation include: raw material insulation temperature of 30° C. to 60° C., vacuum degree of 100 Pa to 2,000 Pa, distillation temperature of 50° C. to 100° C., feeding flow rate of 2 mL/min to 20 mL/min, condensation surface temperature of 0° C. to 30° C., and film scraper speed of 200 rpm to 400 rpm;
(4) adding the first-stage molecular distillation heavy component obtained in step (3) into the molecular distillation device to allow second-stage molecular distillation to remove high-boiling point impurities in the first-stage molecular distillation heavy component, collecting a light component flowing out from a condensation surface of a second-stage molecular distillation device to obtain the SFA and sulforaphene; where the conditions for the second-stage molecular distillation include: raw material insulation temperature of 30° C. to 70° C., vacuum degree of 0.1 Pa to 10 Pa, distillation temperature of 80° C. to 200° C., feeding flow rate of 1 mL/min to 10 mL/min, condensation surface temperature of 0° C. to 20° C., and film scraper speed of 200 rpm to 450 rpm; and
(5) adding 200 g of the SFA and sulforaphene obtained in step (4) into 9.8 kg of corn oil and other vegetable oils to prepare a 2% oil solution of the SFA and sulforaphene (by mass percentage concentration, and percentage contents not otherwise stated below are all mass percentages); stirring the oil solution for 10 min to disperse evenly and reach a turbid state: placing a resulting dispersed oil solution in a refrigerator at 4° C. for 2 h to make the oil solution clear, transparent, and pale yellow; and storing the oil solution in a refrigerator at 4° C. for subsequent use.

In the present disclosure, the medicinal oil can be made into a capsule according to the form of a product, including hard capsules, soft capsules, sustained-release capsules, controlled-release capsules, and enteric-coated capsules, but is not limited thereto.

In the present disclosure, the medicinal oil can be a drug, or a daily chemical such as a health care product, a food, or a hair care product, as long as it can achieve the effect of reducing gastrointestinal reactions caused by SFA and sulforaphene. The use form of the medicinal oil is not limited to the above forms.

In the present disclosure, the medicinal oil may include any pharmaceutically acceptable carrier and/or excipient known in the art, as long as it does not affect the effect of the oil and capsule on reducing gastrointestinal reactions caused by SFA and sulforaphene. For example, lactose, glucose, sucrose, sorbitol, mannitol, starch, dextrin, acacia, calcium phosphate, alginate, gelatin, calcium silicate, microcrystalline cellulose, polyvinylpyrrolidone, water, syrup, methylcellulose, methylparaben, propylparaben, talc, magnesium stearate, super sodium starch glycolate, vegetable oil, animal oil, and mineral oil, but the present disclosure is not limited thereto.

In the present disclosure, in addition to the above ingredients, the medicinal oil may also include other additives, such as lubricants, wetting agents, sweeteners, flavors, emulsifiers, suspending agents, and preservatives. Any additive can be added as long as it does not affect the effect of oil and capsule on reducing gastrointestinal reactions caused by SFA and sulforaphene.

In the present disclosure, the medicinal oil can achieve the effect of reducing gastrointestinal reactions through oral administration, but the administration form is not limited to this.

The present disclosure is further described below with reference to specific examples, but the protection scope the present disclosure is not limited thereto.

### EXAMPLE

### Preparation of SFA and Sulforaphene Extracts

The SFA and sulforaphene extracts were prepared according to the following steps for use in the compositions of the examples described later.

(1) 100 kg of radish seeds were crushed or homogenized, 2,000 L of 20 times a volume of deionized water was added, stirred at room temperature to allow hydrolysis 5 h, hydrochloric acid was added to adjust a pH value of a resulting hydrolyzate to 2.0, allowed to stand overnight, and centrifugation was conducted to obtain 2,000 L of SFA and sulforaphene hydrolyzates as a supernatant; (2) the SFA and sulforaphene hydrolyzates obtained in step (1) were extracted 3 times with 6,000 L of ethyl acetate (2,000 L of the ethyl acetate each time), the SFA and sulforaphene in the hydrolyzate were recovered, 6,000 L in total of an extraction layer in the ethyl acetate was collected, the extraction layer was distilled under reduced pressure at 40° C. and a vacuum degree of −0.08 MPa to obtain 200 L of crude extracts of the SFA and sulforaphene, while the ethyl acetate was recovered; (3) the crude extracts of SFA and sulforaphene obtained in step (2) were added to a molecular distillation device to allow first-stage molecular distillation to remove residual ethyl acetate, moisture, and low-boiling point impurities in the crude extracts of SFA and sulforaphene, a heavy component flowing out from a distillation wall of a first-stage molecular distillation device was collected to obtain 2,000 g of a first-stage molecular distillation heavy component; where the conditions for the first-stage molecular distillation included: raw material insulation temperature of 60° C. vacuum degree of 2,000 Pa, distillation temperature of 100° C. feeding flow rate of 2 mL/min, condensation surface temperature of 0° C., and film scraper speed of 400 rpm; and (4) the first-stage molecular distillation heavy component obtained in step (3) was added into the molecular distillation device to allow second-stage molecular distillation to remove high-boiling point impurities in the first-stage molecular distillation heavy component, a light component flowing out from a condensation surface of a second-stage molecular distillation device was collected to obtain 1,000 g of the SFA and sulforaphene with a high purity of 98%; where the conditions for the second-stage molecular distillation included: raw material insulation temperature of 70° C., vacuum degree of 0.1 Pa, distillation temperature of 110° C., feeding flow rate of 1 mL/min, condensation surface temperature of 110° C., and film scraper speed of 450 rpm.

### Preparation of the Medicinal Oil of the Present Disclosure Containing SFA and Sulforaphene as Active Ingredients

The medicinal oil of the present disclosure was prepared in the form of water-soluble granules, tablets, and capsules by mixing the SFA and sulforaphene extracts prepared by the above method as raw materials with other additives.

### Example 1

### Preparation of Sulforaphene Capsule 1

200 g of the sulforaphene obtained in previous step was added into 9.8 kg of corn oil to prepare a 2% oil solution of the sulforaphene (by mass percentage concentration, and percentage contents not otherwise stated below were all mass percentages). The oil solution was stirred for 10 min to disperse evenly and reach a turbid state. A resulting dispersed oil solution was placed in a refrigerator at 4° C. for 2 h to make the oil solution clear, transparent, and pale yellow. The oil solution was stored in a refrigerator at 4° C. for subsequent use. The glycerin, gelatin, and water were mixed according to a mass ratio of 9:10:1 to obtain a capsule coating, and the capsule coating and the oil solution were prepared by a capsule machine to obtain a sulforaphene soft capsule of 500 mg per grain.

### Example 2

### Preparation of Sulforaphene Capsule 2

100 g of the sulforaphene extract prepared by the above method and 1.9 kg of dextrin were mixed and dissolved in 10 L of deionized water, and then spray-dried in a same manner as that in Example 1 to prepare a spray-dried powder of sulforaphene with a 5% sulforaphene content. The gelatin and water were mixed to obtain a capsule coating, and the capsule coating and the oil solution were prepared by a capsule machine to obtain a sulforaphene hard capsule of 500 mg per grain.

### Example 3

### Preparation of Sulforaphene Capsule 3

200 g of the sulforaphene obtained in previous step was added into 9.8 kg of soybean oil to prepare a 2% oil solution of the sulforaphene. The oil solution was stirred for 10 min to disperse evenly and reach a turbid state. A resulting dispersed oil solution was placed in a refrigerator at 4° C. for 2 h to make the oil solution clear, transparent, and pale yellow. The oil solution was stored in a refrigerator at 4° C. for subsequent use. The glycerin, gelatin, and water were mixed according to a mass ratio of 9:10:1 to obtain a capsule coating, 200 g of cellulose acetate phthalate was added as an enteric coating, and the capsule coating, the enteric coating, and the oil solution were prepared by a capsule machine to obtain a sulforaphene enteric-coated capsule of 500 mg per grain.

### Example 4

### Preparation of Sulforaphene Capsule 4

400 g of the sulforaphene obtained in previous step was added into 9.6 kg of corn oil to prepare a 4% oil solution of the sulforaphene. The oil solution was stirred for 10 min to disperse evenly and reach a turbid state. A resulting dispersed oil solution was placed in a refrigerator at 4° C. for 2 h to make the oil solution clear, transparent, and pale yellow. The oil solution was stored in a refrigerator at 4° C. for subsequent use. The glycerin, gelatin, and water were mixed according to a mass ratio of 9:10:1 to obtain a capsule coating, 200 g of cellulose acetate phthalate was added as an enteric coating, and the capsule coating, the enteric coating, and the oil solution were prepared by a capsule machine to obtain a sulforaphene soft capsule of 500 mg per grain.

### Example 5

### Preparation of Sulforaphene Capsule 5

200 g of the sulforaphene obtained in previous step was added into 9.8 kg of corn oil to prepare a 2% oil solution of the sulforaphene. The oil solution was stirred for 10 min to disperse evenly and reach a turbid state. A resulting dispersed oil solution was placed in a refrigerator at 4° C. for 2 h to make the oil solution clear, transparent, and pale yellow. The oil solution was stored in a refrigerator at 4° C. for subsequent use. The glycerin, gelatin, and water were mixed according to a mass ratio of 9:10:1 to obtain a capsule coating, and the capsule coating and the oil solution were prepared by a capsule machine to obtain a sulforaphene soft capsule of 250 mg per grain.

### Example 6

### Preparation of SFA Capsule 6

400 g of the SFA obtained in previous step was added into 9.6 kg of corn oil to prepare a 4% oil solution of the SFA. The oil solution was stirred for 10 min to disperse evenly and reach a turbid state. A resulting dispersed oil solution was placed in a refrigerator at 4° C. for 2 h to make the oil solution clear, transparent, and pale yellow. The oil solution was stored in a refrigerator at 4° C. for subsequent use. The glycerin, gelatin, and water were mixed according to a mass ratio of 9:10:1 to obtain a capsule coating, 100 g of polyvinylpyrrolidone was added as a controlled-release coating, and the capsule coating, the controlled-release coating, and the oil solution were prepared by a capsule machine to obtain an SFA soft capsule of 500 mg per grain.

### Example 7

### Preparation of SFA Capsule 7

200 g of the SFA obtained in previous step was added into 9.8 kg of corn oil to prepare a 2% oil solution of the SFA. The oil solution was stirred for 10 min to disperse evenly and reach a turbid state. A resulting dispersed oil solution was placed in a refrigerator at 4° C. for 2 h to make the oil solution clear, transparent, and pale yellow: The oil solution was stored in a refrigerator at 4° C. for subsequent use. The glycerin, gelatin, and water were mixed according to a mass ratio of 9:10:1 to obtain a capsule coating, and the capsule coating and the oil solution were prepared by a capsule machine to obtain an SFA soft capsule of 500 mg per grain.

### Example 8

### Preparation of SFA Capsule 8

100 g of the SFA extract prepared by the above method and 1.9 kg of dextrin were mixed and dissolved in 10 L of deionized water, and then spray-dried in a same manner as that in Example 1 to prepare a spray-dried powder of SFA with a 5% SFA content. The gelatin and water were mixed to obtain a capsule coating, and the capsule coating and the oil solution were prepared by a capsule machine to obtain an SFA hard capsule of 500 mg per grain.

1. A medicinal oil for reducing a gastrointestinal reaction caused by sulforaphane (SFA) and sulforaphene, wherein the medicinal oil uses the SFA or/and the sulforaphene serve as active ingredients, the SFA and the sulforaphene each have a weight percentage content of 0.1% to 50%, and the medicinal oil further comprises at least a vegetable oil.

2. The medicinal oil according to claim 1, wherein the SFA and the sulforaphene each are selected from the group consisting of a natural product extract, a biological synthesis product, and a chemical synthesis product.

3. The medicinal oil according to claim 2, wherein the natural product extract is extracted from the group consisting of Brassica oleracea var. gemmifera Zenker, Brassica oleracea var. botrytis Linnaeus, Brassica rapa var. glabra Regel, kale, Brassica oleracea var. acephala DC., broccoli sprouts, Brassica oleracea var. albiflora Kuntze, cauliflower, Brassica juncea var. napiformis Pailleux et Bois, mustard, Brassica rapa L., Raphanus sativus L., Eruca vesicaria subsp. sativa (Miller) Thellung, and Nasturtium officinale R. Br. ex W. T. Aiton.

4. The medicinal oil according to claim 1, wherein the vegetable oil is one or a mixture of two or more selected from the group consisting of soybean oil, corn oil, peanut oil, sesame oil, cottonseed oil, linseed oil, castor oil, rapeseed oil, sunflower seed oil, camellia seed oil, and olive oil.

5. The medicinal oil according to claim 1, wherein the medicinal oil is a capsule, and the capsule is selected from the group consisting of a hard capsule or a soft capsule.

6. The medicinal oil according to claim 5, wherein the capsule is selected from the group consisting of a sustained-release capsule or a controlled-release capsule that is loaded with a sustained-release material and a normal capsule that is not loaded with the sustained-release material.

7. The medicinal oil according to claim 5, wherein the capsule is orally administered.

8. The medicinal oil according to claim 1, further comprising one or more selected from the group consisting of a lubricant, a wetting agent, a sweetener, a flavoring agent, an emulsifier, a suspending agent, and a preservative.

9. A method for treating a gastrointestinal reaction using SFA and sulforaphene.
